(19) United States
(12) Patent Application Publication
(10) Pub. No.: US 2004/0013638 A1
(43) Pub. Date: Jan. 22, 2004

(54) Use of a water-soluble polymer in a biocidal composition for the treatment of hard surfaces

(76) Inventors: Eric Aubay, Le Perreux Sur Marne (FR); Gladys Saliba Gabriel, Alamo, CA (US); Robert Gresser, Vernouillet (FR); Mark Ventura, Freehold, NJ (US); Dominic Yeung, Mississauga (CA)

Correspondence Address: Rhodia Inc., 259 Prospect Plains Road, CN 7500, CRANBURY, NJ, 08512, US

(21) Appl. No.: 10/350,387

(22) Filed: Jan. 23, 2003

Related U.S. Application Data
(63) Continuation of application No. 09/634,316, filed on Aug. 8, 2000, now abandoned.

(30) Foreign Application Priority Data
Aug. 9, 1999 (FR) 99 10329

(51) Int. Cl. A61K 31/765

(52) U.S. Cl. 424/78.37; 424/78.27

(57) ABSTRACT

This invention relates to the use of at least one water-soluble polymer, obtained by copolymerization of at least one monomer (a) with ethylenic unsaturation having a group capable of being protonated in the application medium with at least one monomer with ethylenic unsaturation (b) which is copolymerizable with (a) carrying a functional group with an acidic nature capable of being negatively ionized in the application medium; and optionally at least one monomer with ethylenic unsaturation (c) with a neutral charge, preferably carrying one or more hydrophilic groups, which is copolymerizable with (a) and (b); for improving the biocidal effectiveness of biocidal compositions comprising a cationic biocidal compound.

The subject-matter of the present invention is the treatment of hard industrial, domestic or communal surfaces, in particular of ceramic, tiling or windows type, targeted at conferring biocidal properties on the latter.

The subject-matter of the invention is more particularly the use of a water-soluble polymer in an aqueous biocidal composition, in particular in an aqueous biocidal cleaning composition, for improving the biocidal properties of the latter, by enhancing the adhesion of the biocide to the treated surface. Another subject-matter of the invention is the use, in an aqueous biocidal composition comprising a cationic biocide for the treatment of hard surfaces, of the said water-soluble polymer as agent for the vectorization and/or the controlled release of the said biocide on the hard surface to be treated.

The aqueous biocidal cleaning formulations currently available generally essentially comprise a biocidal compound of cationic nature and a surface-active agent.

However, they do not give entirely satisfactory results for the following reasons:

the interaction of the biocide with the treated surface is weak and does not allow good adhesion or adsorption of the biocide; large amounts of biocidal compound are for this reason necessary in order to confer a true and lasting biocidal activity on the surface;

rinsing of the hard surface after application of the biocidal formulation leads to desorption of the cationic biocide, resulting in a loss of the biocidal properties of the surface;

due to the hydrophobic nature of cationic biocides, their application to hard surfaces has the consequence of conferring a hydrophobic nature on the latter with the consequence of greatly reducing the cleaning performances of the cleaning formulations, in particular in the case of greasy stains.

In order to overcome these problems, provision has been made to add polymer compounds to biocidal cleaning formulations with the aim of improving the biocidal effectiveness of these formulations.

Thus, FR 2,769,469 provides for the combination of a silicone polyether with a cationic biocide in a formulation for cleaning hard surfaces.

GB-2,324,467 discloses the addition of a cationic polymer to a cationic biocide in order to solve the abovementioned problems.

U.S. Pat. No. 5,049,383 discloses aqueous cationic dispersions comprising a biocidal cationic surface-active agent and fine particles of a copolymer comprising at least 80% by weight of units derived from a nonionic ethylenically unsaturated monomer for the antimicrobial, antifungal and algicidal treatment of wood, paint films and the like.

A first aim of the invention is therefore to further improve the biocidal activity of cleaning compositions for hard surfaces.

Another aim of the invention is to improve the adsorption of cationic biocidal substances on hard surfaces, in particular of biocidal substances present in aqueous cleaning compositions.

Yet another aim of the invention is to supply aqueous biocidal cleaning compositions for hard surfaces, in which compositions the amount of cationic biocidal compound(s) is reduced with respect to the existing compositions, which have an effectiveness at least equal to that of the known compositions.

By virtue of the invention, these aims are achieved by the use in aqueous biocidal compositions of a water-soluble amphoteric polymer as described below.

The said polymer in itself does not generally have a biocidal activity.

A subject-matter of the invention is the use of at least one water-soluble amphoteric polymer, obtained by copolymerization:

of at least one monomer (a) with ethylenic unsaturation having a group capable of being protonated in the application medium with

at least one monomer with ethylenic unsaturation (b) which is copolymerizable with (a) carrying a functional group with an acidic nature capable of being negatively ionized in the application medium; and

optionally at least one monomer with ethylenic unsaturation (c) with a neutral charge, preferably carrying one or more hydrophilic groups, which is copolymerizable with (a) and (b); for improving the biocidal effectiveness of aqueous biocidal compositions comprising a cationic biocidal compound.

The monomer (a) advantageously comprises at least one quaternary ammonium group.

The monomer (a) is preferably chosen from the compounds of following general formulae I to III:

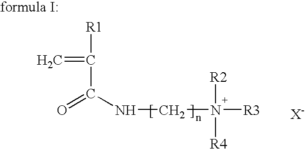

in which

R₁ is a hydrogen atom or a methyl group, preferably a methyl group;

R₂, R₃ and R₄ are linear or branched C₁-C₄ alkyl groups;

n represents an integer from 1 to 4, in particular the number 3;

X represents a counterion compatible with the water-soluble nature of the polymer;

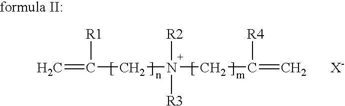

in which:

R₁ and R₄ represent, independently of one another, a hydrogen atom or a linear or branched C₁-C₆ alkyl group;

R₂ and R₃ represent, independently of one another, an alkyl, hydroxyalkyl or aminoalkyl group in which the alkyl group is a linear or branched C₁-C₆ chain, preferably a methyl group;

n and m are integers between 1 and 3;

X represents a counterion compatible with the water-soluble nature of the polymer;

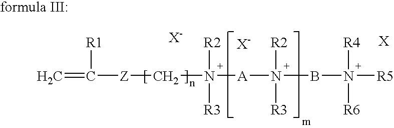

in which

R₁ is a hydrogen atom or a methyl or ethyl group;

R₂, R₃, R₄, R₅ and R₆, which are identical or different, are linear or branched C₁-C₆ alkyl, hydroxyalkyl or aminoalkyl groups;

m is an integer from 0 to 10, preferably from 0 to 2;

n is an integer from 1 to 6, preferably 2 to 4;

Z represents a —C(O)O— or —C(O)NH— group or an oxygen atom;

A represents a (CH₂)ₚ group, p being an integer from 1 to 6, preferably from 2 to 4;

B represents a linear or branched C₂-C₁₂, advantageously C₃-C₆, polymethylene chain optionally interrupted by one or more heteroatoms or heterogroups, in particular O or NH, and optionally substituted by one or more hydroxyl or amino groups, preferably hydroxyl groups;

X, which are identical or different, represent counterions compatible with the water-soluble nature of the polymer.

Preference is given, as monomer (a) of general formula I, to that represented by the following formula:

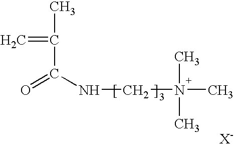

in which the counterion is a chloride (MAPTAC).

Preference is given, as monomer (a) of general formula II, to that represented by the following formula:

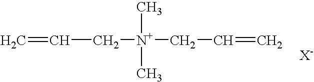

in which X is a chloride (DADMAC).

Preference is given, in the general formula III, to those which fulfil the following conditions:

Z represents C(O)O, C(O)NH or O, very preferably C(O)NH;

n is equal to 2 or 3, very particularly 3;

m ranges from 0 to 2 and is preferably equal to 0 or 1, very particularly 0;

B represents

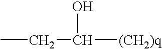

with q from 1 to 4, preferably equal to 1;

R₁ to R₆, which are identical or different, represent a methyl or ethyl group.

Preferred monomers (a) of this type are those of following formula:

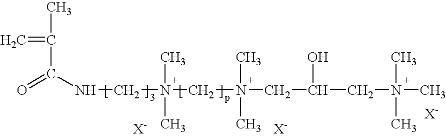

p=2 to 4,

and more particularly the monomer

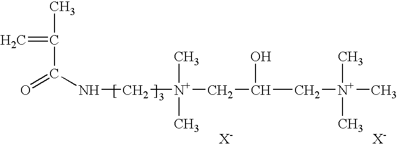

X⁻ representing the chloride ion (Diquat).

Advantageously, (b) is chosen from C₃-C₈ carboxylic, sulphonic, sulphuric, phosphonic and phosphoric acids with monoethylenic unsaturation.

The monomer (b) is preferably chosen from acrylic acid, methacrylic acid, α-ethacrylic acid, β,β-dimethylacrylic acid, methylenemalonic acid, vinylacetic acid, allylacetic acid, ethylideneacetic acid, propylideneacetic acid, crotonic acid, maleic acid, fumaric acid, itaconic acid, citraconic acid, mesaconic acid, N-(methacryloyl)alanine, N-(acryloyl)hydroxyglycine, sulphopropyl acrylate, sulphoethyl acrylate, sulphoethyl methacrylate, styrenesulphonic acid, vinylsulphonic acid, vinylphosphonic acid, phosphoethyl acrylate, phosphonoethyl acrylate, phosphopropyl acrylate, phosphonopropyl acrylate, phosphoethyl methacrylate, phosphonoethyl methacrylate, phosphopropyl methacrylate, phosphonopropyl methacrylate and the alkali metal and ammonium salts of the latter.

The monomer (c) is advantageously chosen from acrylamide, vinyl alcohol, C₁-C₄ alkyl esters of acrylic acid and of methacrylic acid, C₁-C₄ hydroxyalkyl esters of acrylic acid and of methacrylic acid, in particular ethylene glycol and propylene glycol acrylate and methacrylate, polyalkoxylated esters of acrylic acid and of methacrylic acid, in particular the polyethylene glycol and polypropylene glycol esters, esters of acrylic acid or of methacrylic acid and of polyethylene glycol or polypropylene glycol C₁-C₂₅ monoalkyl ethers, vinyl acetate, vinylpyrrolidone or methyl vinyl ether.

In the above formulae I, II and III, X is advantageously chosen from halogen, in particular chlorine, sulphonate, sulphate, hydrogensulphate, phosphate, phosphonate, citrate, formate and acetate anions.

Generally, the level of monomer (a) is advantageously between 3 and 80 mol %, preferably 10 to 70 mol %. The level of monomer (b) is advantageously between 10 and 95 mol %, preferably 20 to 80 mol %. The level of monomer (c) is advantageously between 0 and 50 mol %, preferably 0 and 30 mol %, very particularly from 5 to 25 mol %, this level being such that the polymer formed is soluble in the aqueous application medium.

The molar ratio of cationic monomer to the anionic monomer (a)/(b) is advantageously between 80/20 and 5/95, preferably between 75/25 and 20/80.

The molecular mass of the water-soluble polymer according to the invention is at least 1000, advantageously at least 10,000, and at most 20,000,000, advantageously at most 10,000,000.

Except when otherwise indicated, when the term molecular mass is used, it will refer to the weight-average molecular mass, expressed in g/mol. The latter can be determined by aqueous gel permeation chromatography (GPC) or measurement of the intrinsic viscosity in a 1N NaNO₃ solution at 30° C.

The copolymer is preferably random.

The monomers of general formulae I and II are known or can easily be prepared using processes well known in the art.

The monomer of general formula III can be prepared, for example, according to the following reaction schemes:

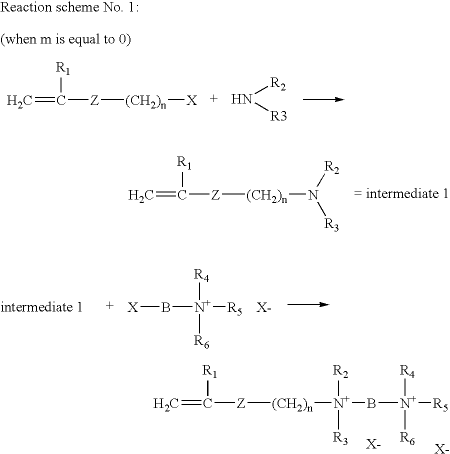

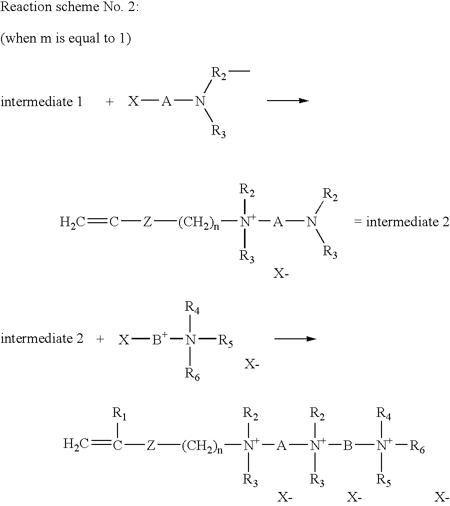

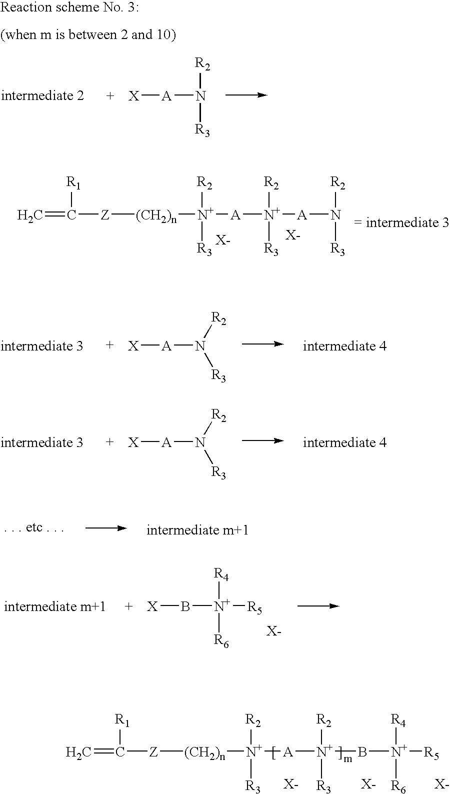

The copolymers of the invention can be obtained according to known techniques for the preparation of copolymers, in particular by polymerization by the radical route of the starting ethylenically unsaturated monomers, which are known compounds or compounds which can be easily obtained by a person skilled in the art by employing conventional synthetic processes of organic chemistry.

Reference may in particular be made to the processes disclosed in U.S. Pat. No. 4,387,017 and EP 156,646.

The radical polymerization is preferably carried out in an environment which is devoid of oxygen, for example in the presence of an inert gas (helium, argon, and the like) or of nitrogen. The reaction is carried out in an inert solvent, preferably ethanol or methanol, and more preferably in water.

The polymerization is initiated by addition of a polymerization initiator. The initiators used are the free radical initiators commonly used in the art. Examples comprise organic peresters (t-butylperoxy pivalate, t-amylperoxy pivalate, t-butylperoxy a-ethylhexanoate, and the like); organic compounds of azo type, for example azobisamidinopropane hydrochloride, azobisisobutyronitrile, azobis(2,4-dimethylvaleronitrile), and the like; inorganic and organic peroxides, for example hydrogen peroxide, benzyl peroxide and butyl peroxide, and the like; redox initiating systems, for example those comprising oxidizing agents, such as persulphates (in particular ammonium or alkali metal persulphates, and the like); chlorates and bromates (including inorganic or organic chlorates and/or bromates); reducing agents, such as sulphites and bisulphites (including inorganic and/or organic sulphites or bisulphites); oxalic acid and ascorbic acid, as well as the mixtures of two or more of these compounds.

The preferred initiators are water-soluble initiators. Sodium persulphate and azobisamidinopropane hydrochloride are in particular preferred.

In an alternative form, the polymerization can be initiated by irradiation using ultraviolet light. The amount of initiator used is generally an amount sufficient to produce initiation of the polymerization. The initiators are preferably present in an amount ranging from 0.001 to approximately 10% by weight with respect to the total weight of the monomers and are preferably in an amount of less than 0.5% by weight with respect to the total weight of the monomers, a preferred amount being situated in the range from 0.005 to 0.5% by weight with respect to the total weight of the monomers. The initiator is added to the polymerization mixture either continuously or noncontinuously.

When it is wished to obtain copolymers of high molecular mass, it is desirable to add fresh initiator during the polymerization reaction. The gradual or noncontinuous addition also makes possible a more efficient polymerization and a shorter reaction time. The polymerization is carried out under reaction conditions which are effective in polymerizing the monomers (a), the monomers (b) and optionally the monomers (c) under an atmosphere devoid of oxygen. The reaction is preferably carried out at a temperature ranging from approximately 30° C. to approximately 100° C. and preferably between 60° C. and 90° C. The atmosphere which is devoid of oxygen is maintained throughout the duration of the reaction, for example by maintaining a nitrogen flow throughout the reaction.

The following are preferred water-soluble amphoteric copolymers:

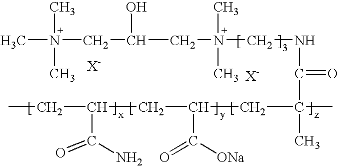

derivative of Diquat, of acrylic acid (sodium salt) and of acrylamide;

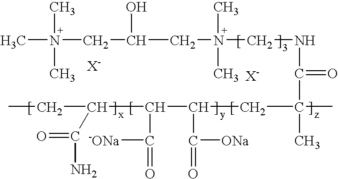

derivative of Diquat, of maleic acid (sodium salt) and of acrylamide;

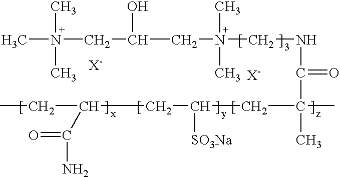

derivative of Diquat, of vinylsulphonic acid (sodium salt) and of acrylamide;

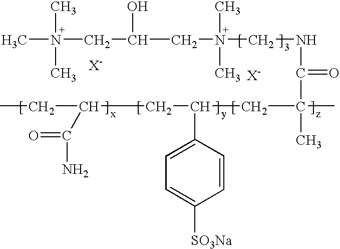

derivative of Diquat, of styrenesulphonic acid (sodium salt) and of acrylamide;

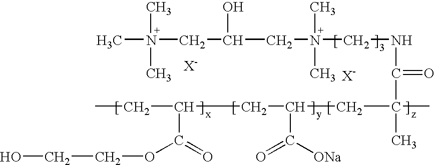

derivative of Diquat, of acrylic acid (sodium salt) and of hydroxyethyl acrylate;

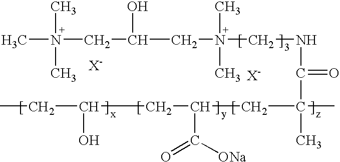

derivative of Diquat, of acrylic acid (sodium salt) and of vinyl alcohol;

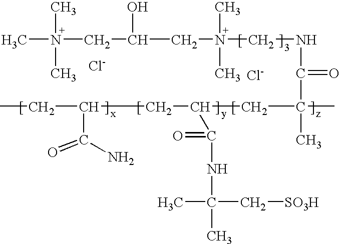

derivative of Diquat, of N-(1-sulpho-2-isobutyl)-acrylamide and of acrylamide;

x having a mean value of 0 to 50%, preferably of 0 to 30%, very particularly of 5 to 25%,

y having a mean value of 10 to 95%, preferably of 20 to 70%,

z having a mean value of 3 to 80%, preferably of 10 to 60%,

and the y/z ratio preferably being of the order of 4/1 to 1/3,

with x+y+z=100%, x, y and z representing the mol % of each unit derived from each of the monomers (c), (b) and (a),

and

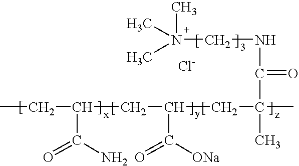

derivative of MAPTAC, of acrylic acid (sodium salt) and of acrylamide;

with x+y+z=100%, x, y and z representing the mol % of each unit derived from each of the monomers (c), (b) and (a)

y/z=25/75 to 70/30

and x having a mean value of 0 to 40%, preferably of 10 to 30%.

Mention may also be made of the MAPTAC copolymers of above formula in which the units derived from acrylic acid are replaced by units derived from maleic acid, vinylsulphonic acid, styrenesulphonic acid (sodium salts) or N-(1-sulpho-2-isobutyl)acrylamide; likewise, the units derived from acrylamide can be replaced by units derived from vinyl alcohol or from hydroxyethyl acrylate.

Other advantageous copolymers are those derived from DADMAC with the same formula as those above derived from MAPTAC.

All cationic biocides are suitable for the purposes of the invention. The biocide is preferably chosen from:

quaternary monoammonium salts of formulae

R¹R²R³R⁴N⁺X⁻

where

R¹ represents a benzyl group optionally substituted by a chlorine atom or a C₁-C₄ alkylbenzyl group,

R² represents a C₈-C₂₄ alkyl group,

R³ and R⁴, which are alike or different, represent a C₁-C₄ alkyl or hydroxyalkyl group,

X⁻ is a solubilizing anion, such as halide (for example, chloride, bromide or iodide), sulphate or methyl sulphate;

R^{1′}R^{2′}R^{3′}R^{4′}N⁺X⁻

where

R^{1′} and R^{2′}, which are alike or different, represent a C₈-C₂₄ alkyl group,

R^{3′} and R^{4′}, which are alike or different, represent a C₁-C₄ alkyl group,

X⁻ is a solubilizing anion, such as halide (for example, chloride, bromide or iodide), sulphate or methyl sulphate;

R^{1″}R^{2″}R^{3″}R^{4″}N⁺X⁻

where

R^{1″} represents a C₈-C₂₄ alkyl group,

R^{2″}, R^{3″}and R^{4″}, which are alike or different, represent a C₁-C₄ alkyl group,

X⁻ is a solubilizing anion, such as halide (for example, chloride, bromide or iodide), sulphate or methyl sulphate; in particular:

A cocoalkylbenzyldimethylammonium, (C₁₂-C₁₄ alkyl)benzyldimethylammonium, cocoalkyl(dichlorobenzyl)dimethylammonium, tetradecylbenzyldimethylammonium, didecyldimethylammonium or dioctyldimethylammonium chlorides,

monoquaternary heterocyclic amine salts, such as laurylpyridinium, cetylpyridinium or (C₁₂-C₁₄ alkyl)benzylimidazolium chlorides;

(fatty alkyl)triphenylphosphonium salts, such as myristyltriphenylphosphonium bromide;

amphoteric biocides, such as N-[N′-(N″-(C₈-C₁₈ alkyl)-3-aminopropyl]glycine, N-[N′-(N″-(C₈-C₁₈ alkyl)-2-aminoethyl)-2-aminoethyl]glycine or N,N-bis[N′-(C₈-C₁₈ alkyl)-2-aminoethyl]glycine derivatives, such as (dodecyl)(aminopropyl)glycine or (dodecyl)(diethylenediamine)glycine;

amines, such as N-(3-aminopropyl)-N-dodecyl-1,3-propanediamine.

The use of the polymers of the invention in formulae comprising a cationic biocide in the treatment of hard surfaces increases the effectiveness of the biocide and limits the negative effects encountered with this type of formula.

This is because the cationic groups (a) of the polymer make possible a significant and persistent adsorption of the polymer on the hard surface, which is generally negatively charged.

The anionic groups (b) of the polymer interact with the cationic biocide and make possible good anchoring of the biocide to the hard surface.

It should be noted that, in the absence of polymer, the cationic biocides can also interact with the hard surface but this interaction is markedly greater with a polymer, by virtue of the collaborative effect of the polymer/surface bonds.

The joint use of a cationic biocide and of the polymer of the invention thus introduce the following advantages:

the polymer according to the invention makes it possible to vectorize the biocide onto the hard surface; this makes possible anchoring of the biocide to the surface to be treated and increases the effectiveness of the biocide;

the polymer according to the invention remains present after successive rinsing operations and makes possible persistent adsorption of the biocide on the surface; this makes it possible to obtain long-term effectiveness of the biocide;

the polymer according to the invention furthermore makes it possible to substantially decrease the amount of biocide needed to obtain good biocidal activity on the treated surface.

A second object of the invention consists of the use, in an aqueous biocidal composition comprising a cationic biocide for the treatment of hard surfaces, of the said water-soluble polymer as agent for the vectorization and/or the controlled release of the said biocide on the hard surface to be treated.

In addition to the cationic biocidal compound and the water-soluble polymer according to the invention, the composition for the treatment of hard surfaces can in particular comprise a surface-active agent.

Nonionic surface-active agents are preferred.

Mention may be made, among nonionic surface-active agents, of in particular alkylene oxide, especially ethylene oxide, condensates with alcohols, polyols, alkylphenols, fatty acid esters, fatty acid amides and fatty amines; amine oxides; sugar derivatives, such as alkylpolyglycosides or esters of fatty acids and of sugars, in particular sucrose monopalmitate; tertiary phosphine oxides with a long chain; dialkyl sulphoxides; sequential copolymers of polyoxyethylene and of polyoxypropylene; polyalkoxylated sorbitan esters; sorbitan fatty esters, polyethylene oxide)s and amides of fatty acids which are modified so as to confer on them a hydrophobic nature (for example, the mono- and diethanolamides of fatty acids comprising from 10 to 18 carbon atoms).

Mention may very particularly be made of

polyoxyalkylenated (polyoxyethylenated, polyoxypropylenated or polyoxybutylenated) alkylphenols with a C₆-C₁₂ alkyl substituent and comprising from 5 to 25 oxyalkylene units; mention may be made, by way of example, of Triton X-45, Triton X-114, Triton X-10O or Triton X-102, sold by Rohm & Haas Co.;

glucosamides, glucamides or glycerolamides;

polyoxyalkylenated C₈-C₂₂ aliphatic alcohols comprising from 1 to 25 oxyalkylene (oxyethylene or oxypropylene) units. Mention may be made, by way of example, of Tergitol 15-S-9 or Tergitol 24-L-6 NMW, sold by Union Carbide Corp., Neodol 45-9, Neodol 23-65, Neodol 45-7 or Neodol 45-4, sold by Shell Chemical Co., or Rhodasurf ID060, Rhodasurf LA90 or Rhodasurf IT070, sold by the company Rhodia.

amine oxides, such as (C₁₀-C₁₈ alkyl)-dimethylamine oxides or (C₈-C₂₂ alkoxy)-ethyldihydroxyethylamine oxides;

the alkylpolyglycosides disclosed in U.S. Pat. No. 4,565,647;

C₈-C₂₀ fatty acid amides;

ethoxylated fatty acids;

ethoxylated amines.

Another subject-matter of the invention is the use as defined above, characterized in that a composition comprising:

a cationic biocidal compound;

a water-soluble polymer as defined above;

a nonionic surfactant, is applied.

The composition advantageously comprises:

from 0.1 to 10%, preferably from 0.3 to 5%, by weight of a cationic biocide;

from 0.01 to 3%, preferably 0.05 to 2%, by weight of a water-soluble polymer as defined above;

from 0.5 to 15%, preferably from 1 to 10%, by weight of a nonionic surfactant.

The cleaning composition according to the invention is applied to the surface to be treated in an amount such that it allows, after rinsing, if appropriate, and after drying, a deposition of copolymer according to the invention of 0.0001 to 1 g/m², preferably 0.001 to 0.1 g/m², of surface to be treated.

According to the invention, in addition to the biocide and the copolymer according to the invention, which are the main constituents of the aqueous biocidal system of the invention, it is advantageously possible for other constituents to be present, such as chelating agents (for example aminocarboxylates (ethylenediaminetetraacetates, nitrilotriacetates or N,N-bis(carboxymethyl)glutamates) or citrates), alcohols (ethanol, isopropanol or glycols), detergency adjuvants (phosphates or silicates), dyes, fragrances, and the like.

The said biocidal cleaning composition can be employed for disinfecting floors, walls, work surfaces, equipment, furniture, instruments, and the like in industry, the food processing field, the domestic sphere (kitchens, bathrooms, and the like) and communally.

Mention may be made, among the surfaces which can be treated, of those made of ceramic, glass, poly(vinyl chloride), formica or other hard organic polymer, stainless steel, aluminium, wood, and the like.

The cleaning and disinfecting operation consists in applying the said biocidal cleaning composition, optionally diluted by 1 to 1000 times, preferably by 1 to 100 times, to the hard surface to be treated.

The amount of biocidal system which can be favourably employed is that corresponding to a deposition of 0.01 to 10 g, preferably of 0.1 to 1 g, of biocide per m² of surface and to a deposition of 0.001 to 2 g, preferably of 0.01 to 0.5 g, of copolymer of the invention per m² of surface.

Mention may be made, among the microorganisms whose proliferation can be controlled by employing the biocidal cleaning composition of the invention, of

A Gram negative bacteria, such as: Pseudomonas aeruginosa; Escherichia coli; Proteus mirabilis

A Gram positive bacteria, such as: Staphylococcus aureus; Streptococcus faecium

A other bacteria which are dangerous in food, such as: Salmonella typhimurium; Listeria monocytogenes; Campylobacter jejuni; Yersinia enterocolitica

A yeasts, such as: Saccharomyces cerevisiae; Candida albicans

A fungi, such as: Aspergillus niger; Fusarium solani; Pencillium chrysogenum

A algae, such as: Chlorella saccharophilia; Chlorella emersonii; Chlorella vulgaris; Chlamydomonas eugametos.

The biocidal system of the invention is very particularly effective against the Gram negative microorganism Pseudomonas aeroginosa, the Gram positive microorganism Staphylococcus aureus or the fungus Aspergillus niger.

The following examples illustrate the invention.

### EXAMPLES 1 to 5

Test of Disinfection of a Hard Surface

1) Aqueous Biocidal Solutions Tested

The following aqueous biocidal solutions are prepared:

solution composed of:

| | |
|---|---|
| Rhodaquat RP 50* | 3% (i.e., 1.5% of biocidal |
| | active material) |
| +nonionic surfactant | 5% (C₁₀ alcohol with 6 ethylene |
| | oxide units) |
| +polymer | 0 or 0.15% |

*(C₁₂-C₁₄ alkyl) benzyldimethylammonium chloride.

These solutions are subsequently diluted 60 times in order to carry out the test.

The polymers evaluated during these trials have the following structures:

Polymer 1:

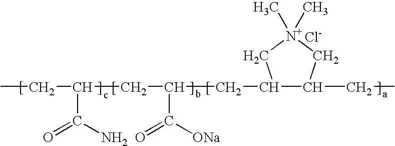

with a=4, b=4, c=2

Polymers 2 to 4:

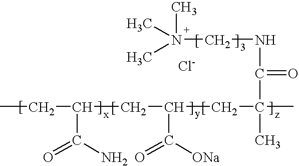

Polymer 2:

x=2, y=4, z=4

Polymer 3:

x=2, y=6, z=2

Polymer 4:

x=0, y=7, z=3

Polymer 5:

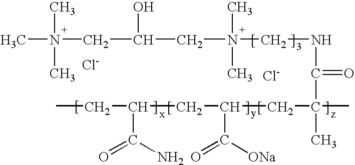

with x=2, y=4, z=4

Polymer 5 is prepared as follows:

The following ingredients are added to a 1 litre reactor:

| | |
|---|---|
| Demineralized water | 633 |
| 52% Acrylamide | 29.3 |
| Acrylic acid | 30.9 |
| 65% Diquat monomer | 236.7 |
| Versene 100 | 0.2 |

(EDTA from Dow Chemical)

The mixture obtained is heated gently to 75° C. at a pH of approximately 2.6 under a gentle nitrogen flow. After 30 minutes, when the temperature reaches 75° C., an initiator solution based on sodium persulphate (0.1 g in 1.0 g of demineralized water) is added to the reactor in a single step. Cooling is necessary in order to keep the temperature at 75° C. and the mixture becomes viscous after approximately 45 minutes. Two additional portions of initiating solution based on persulphate are added after reacting for one and two hours respectively. The reaction mixture is subsequently heated to a temperature of 85° C. and maintained at this temperature for an additional two hours before being cooled to 25° C. The viscosity of the resulting solution of Polymer 1 is approximately 29,500 cPs with a total content of solids of approximately 20.5%. The pH of the 10% solution is approximately 2.2. The residual acrylamide is less than 0.1% by weight.

2) Method of Carrying Out the Test on a White Ceramic Tile

1. 3 g of dilute aqueous biocidal solution are added to the surface of the ceramic tile (5 cm×5 cm) sterilized beforehand by cleaning with isopropyl alcohol. The tile is dried at 45° C. in an oven.

2. The surface of the tile is positioned vertically and is sprayed with one gram of water using a hand sprayer. This corresponds to a washing operation without mechanical action. Between 0 and 15 washing operations are thus carried out before drying at 45° C.

3. 0.25 ml of an aqueous medium comprising approximately 108 CFU/ml of Gram negative bacterium, Pseudomonas aeruginosa, is added and is spread over the pretreated hard surface.

4. The tile is left at room temperature for 3 hours, in order to allow the biocide to migrate from the surface of the polymer and to kill the surface bacteria.

5. The tile is dried at 37° C. for at least 30 minutes.

6. The surviving microorganisms are recovered by using a sterile cottonwool pad moistened beforehand with a neutralizing solution. The entire surface is carefully cleaned by wiping 4 times in all directions.

7. The pad is introduced into 9 ml of neutralizing medium; the volume is adjusted to 10 ml with water. The bacterial suspension is transferred onto Nutrient Agar in Petri dishes by successive dilutions by a factor 10.

8. The dishes are incubated at 37° C. for 48 hours and the surviving microorganisms are counted.

The neutralizing medium comprises 3% of Tween 80 polysorbate and 2% of soybean lecithin.

A control test is performed by carrying out Stages 1. to 7. on the surface of a white ceramic tile (5 cm×5 cm) which has been sterilized beforehand but which has not been treated with the biocidal system.

The log₁₀ for reduction of the number of bacteria is calculated as follows:

log₁₀ for reduction=log₁₀ N/n

N being the number of surviving bacteria (in CFU/ml) in the control test

n being the number of surviving bacteria (in CFU/ml) in the test employing the biocidal system.

3) Results

The results of the test carried out above appear in Table 1.

The results of Example 6, given by way of comparison, show that an aqueous solution of biocidal agent alone does not withstand the 15 rinsing operations.

The results of Example 7, given by way of comparison, show that the polymers 1 to 5 in themselves do not have a biocidal property.

The results of Examples 1 to 5 show that the interaction between the biocide and the polymer introduces long-term protection of the surface against bacteria, without damaging the short-term bactericidal performances.

| | | Log₁₀ for | Log₁₀ for |
|---|---|---|---|
| | | reduction | reduction |
| | | after 0 | after 15 |
| | | washing | washing |
| Example | Polymer | operations | operations |
| 1 | Polymer 1: 0.15% | 6 | 6 |
| 2 | Polymer 2: 0.15% | 6 | 6 |
| 3 | Polymer 3: 0.15% | 6 | 6 |
| 4 | Polymer 4: 0.15% | 6 | 6 |
| 5 | Polymer 5: 0.15% | 6 | 6 |
| 6 | Without polymer | 6 | 0 |
| 7 | Without biocide | 0 | 0 |
| | and with 0.15% of | | |
| | Polymer 1 to 5 | | |

1. Use of at least one water-soluble polymer, obtained by copolymerization:
of at least one monomer (a) with ethylenic unsaturation having a group capable of being protonated in the application medium with
at least one monomer with ethylenic unsaturation (b) which is copolymerizable with (a) carrying a functional group with an acidic nature capable of being negatively ionized in the application medium; and
optionally at least one monomer with ethylenic unsaturation (c) with a neutral charge, preferably carrying one or more hydrophilic groups, which is copolymerizable with (a) and (b); for improving the biocidal effectiveness of aqueous biocidal compositions comprising a cationic biocidal compound.

2. Use according to claim 1, characterized in that the monomer (a) comprises at least one quaternary ammonium group.

3. Use according to claim 1 or claim 2, in which the monomer (a) is chosen from the compounds of following general formulae I, II and III: 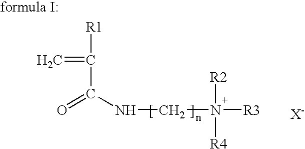
in which
R₁ is a hydrogen atom or a methyl group, preferably a methyl group;
R₂, R₃ and R₄ are linear or branched C₁-C₄ alkyl groups;
n represents an integer from 1 to 4, in particular the number 3;
X represents a counterion compatible with the water-soluble nature of the polymer; 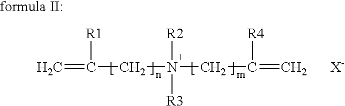
in which:
R₁ and R₄ represent, independently of one another, a hydrogen atom or a linear or branched C₁-C₆ alkyl group;
R₂ and R₃ represent, independently of one another, an alkyl, hydroxyalkyl or aminoalkyl group in which the alkyl group is a linear or branched C₁-C₆ chain, preferably a methyl group;
n and m are integers between 1 and 3;
X represents a counterion compatible with the water-soluble nature of the polymer; 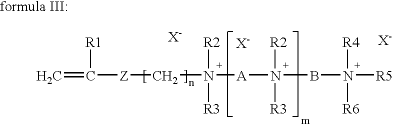
in which
R₁ is a hydrogen atom or a methyl or ethyl group;
R₂, R₃, R₄, R₅ and R₆, which are identical or different, are linear or branched C₁-C₆ alkyl, hydroxyalkyl or aminoalkyl groups;
m is an integer from 0 to 10, preferably from 0 to 2;
n is an integer from 1 to 6, preferably 2 to 4;
Z represents a —C(O)O— or —C(O)NH— group or an oxygen atom;
A represents a (CH₂)ₚ group, p being an integer from 1 to 6, preferably from 2 to 4;
B represents a linear or branched C₂-C₁₂, advantageously C₃-C₆, polymethylene chain optionally interrupted by one or more heteroatoms or heterogroups, in particular O or NH, and optionally substituted by one or more hydroxyl or amino groups, preferably hydroxyl groups;
X, which are identical or different, represent counterions compatible with the water-soluble nature of the polymer.

4. Use according to claim 3, characterized in that the monomer (a) is represented by the following formula: 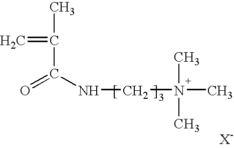
X⁻ being as defined in claim 1, preferably a chloride (MAPTAC).

5. Use according to claim 3, characterized in that the monomer (a) is represented by the following formula: 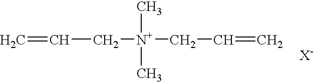
X⁻ being as defined in claim 1, preferably a chloride (DADMAC).

6. Use according to claim 3, characterized in that the monomer (a) is represented by the general formula III according to claim 5, in which
Z represents C(O)O, C(O)NH or O, very preferably C(O)NH;
n is equal to 2 or 3, very particularly 3;
m ranges from 0 to 2 and is preferably equal to 0 or 1, very particularly 0;
B represents 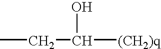
with q from 1 to 4, preferably equal to 0.1;
R₁ to R₆, which are identical or different, represent a methyl or ethyl group.

7. Use according to claim 6, characterized in that the monomer (a) is represented by the following formula: 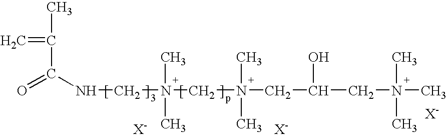
p=2 to 4.

8. Use according to claim 7, characterized in that the monomer (a) is: 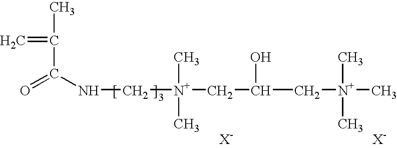
X⁻ representing the chloride ion (Diquat).

9. Use according to any one of the preceding claims, characterized in that (b) is chosen from C₃-C₈ carboxylic, sulphonic, sulphuric, phosphonic and phosphoric acids with monoethylenic unsaturation.

10. Use according to claim 9, characterized in that the monomer (b) is chosen from acrylic acid, methacrylic acid, α-ethacrylic acid, β,β-dimethylacrylic acid, methylenemalonic acid, vinylacetic acid, allylacetic acid, ethylideneacetic acid, propylideneacetic acid, crotonic acid, maleic acid, fumaric acid, itaconic acid, citraconic acid, mesaconic acid, N-(methacryloyl)alanine, N-(acryloyl)hydroxyglycine, sulphopropyl acrylate, sulphoethyl acrylate, sulphoethyl methacrylate, styrenesulphonic acid, vinylsulphonic acid, vinylphosphonic acid, phosphoethyl acrylate, phosphonoethyl acrylate, phosphopropyl acrylate, phosphonopropyl acrylate, phosphoethyl methacrylate, phosphonoethyl methacrylate, phosphopropyl methacrylate, phosphonopropyl methacrylate and the alkali metal and ammonium salts of the latter.

11. Use according to any one of the preceding claims, characterized in that the monomer (c) is chosen from acrylamide, vinyl alcohol, C₁-C₄ alkyl esters of acrylic acid and of methacrylic acid, C₁-C₄ hydroxyalkyl esters of acrylic acid and of methacrylic acid, in particular ethylene glycol and propylene glycol acrylate and methacrylate, polyalkoxylated esters of acrylic acid and of methacrylic acid, in particular the polyethylene glycol and polypropylene glycol esters, esters of acrylic acid or of methacrylic acid and of polyethylene glycol or polypropylene glycol C₁-C₂₅ monoalkyl ethers, vinyl acetate, vinylpyrrolidone or methyl vinyl ether.

12. Use according to any one of the preceding claims, characterized in that, in the general formulae I, II and/or III, X is chosen from halogen, in particular chlorine, sulphonate, sulphate, hydrogensulphate, phosphate, phosphonate, citrate, formate and acetate anions.

13. Use according to any one of the preceding claims, characterized in that the water-soluble copolymer is obtained by copolymerization:
of 3 to 80 mol %, preferably 10 to 70 mol %, of the monomer (a);
of 10 to 95 mol %, preferably 20 to 80 mol %, of the monomer (b);
of 0 to 50 mol %, preferably of 5 to 30 mol %, of the monomer (c);
the level of optional monomer (c) being such that the polymer is soluble in the aqueous application medium.

14. Use according to any one of the preceding claims, characterized in that the (a)/(b) molar ratio is between 80/20 and 5/95 and preferably between 75/25 and 20/80.

15. Use according to any one of the preceding claims, characterized in that the molecular mass of the copolymer is at least 1000, advantageously at least 10,000, and at most 20,000,000, advantageously at most 10,000,000.

16. Use according to claim 1, characterized in that the polymer is chosen from the following compounds:
MAPTAC copolymers 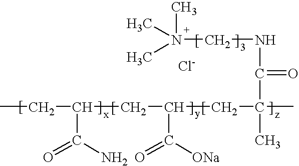
with x+y+z=100%, x, y and z representing the mol % of each unit derived from each of the monomers (c), (b) and (a)
y/z=25/75 to 70/30
and x having a mean value of 0 to 40%, preferably of 10 to 30%,
and homologous copolymers in which the monomer (b), acrylic acid (sodium salt), and/or (c), acrylamide, is replaced by a different monomer (b) chosen from maleic acid, vinylsulphonic acid, styrenesulphonic acid (sodium salts) or N-(1-sulpho-2-isobutyl)acrylamide and/or a different monomer (c) chosen from vinyl alcohol or hydroxyethyl acrylate
DADMAC copolymers homologous with the preceding MAPTAC copolymers, in which the MAPTAC is replaced by DADMAC as monomer (a)
Diquat copolymers 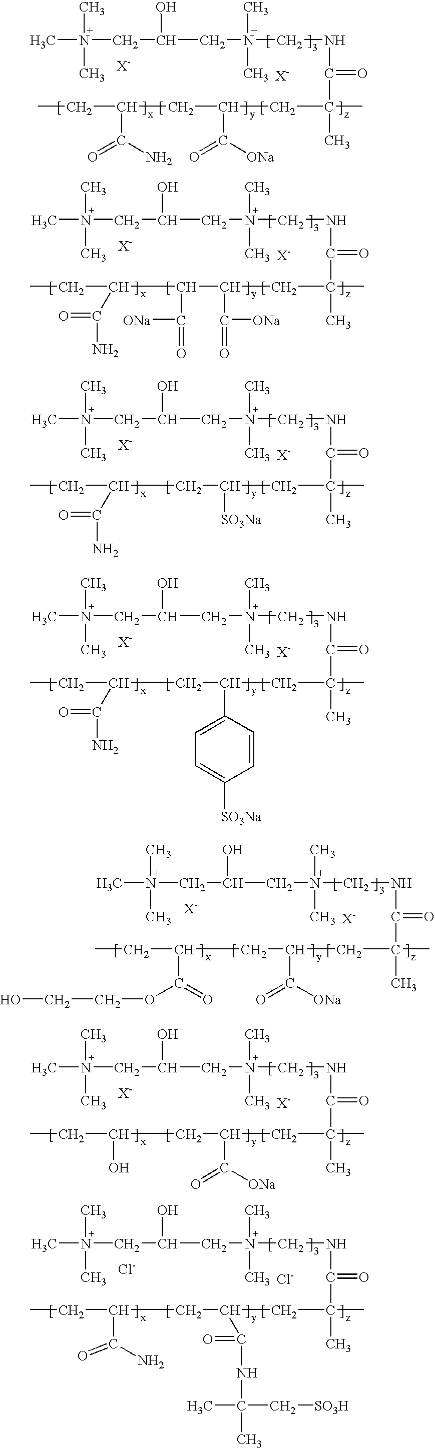
with x having a mean value of 0 to 50%, preferably of 0 to 30%, very particularly of 5 to 25%,
y having a mean value of 10 to 95%, preferably of 20 to 70%,
z having a mean value of 3 to 80%, preferably of 10 to 60%,
and the y/z ratio preferably being of the order of 4/1 to 1/3,
with x+y+z=100%, x, y and z representing the mol % of each unit derived from each of the monomers (c), (b) and (a).

17. Use according to any one of the preceding claims, characterized in that the cationic biocidal compound is chosen from:
quaternary monoammonium salts;
monoquaternary heterocyclic amine salts;
(fatty alkyl)triphenylphosphonium salts;
polymeric biocides.

18. Use according to any one of the preceding claims, characterized in that the said biocidal composition additionally comprises a nonionic surface-active agent.

19. Use according to claim 23, characterized in that the composition comprises:
from 0.1 to 10%, preferably from 0.3 to 5%, by weight of a cationic biocide;
from 0.01 to 3%, preferably 0.05 to 2%, by weight of a water-soluble polymer as defined in claims 1 to 16;
from 0.5 to 15%, preferably from 1 to 10%, by weight of a nonionic surfactant.

20. Use according to any one of claims 1 to 19, for the biocidal treatment of hard surfaces.

21. Use, in an aqueous biocidal composition comprising a cationic biocide for the treatment of hard surfaces, of the water-soluble polymer as defined in claims 1 to 16 as agent for the vectorization and/or the controlled release of the said biocide on the hard surface to be treated.
